(19) United States
(12) Patent Application Publication
(10) Pub. No.: US 2008/0003608 A1
(43) Pub. Date: Jan. 3, 2008

(54) Highly Specific Polyclonal Antibodies of Individual Core Histone and Uses Thereof

(76) Inventors: Kandan Kulangara Febitha, Madhya Pradesh State (IN); Ashish Varier Radhika, Tamil Nadu State (IN); Kumar Kundu Tapas, Karnataka State (IN); Das Chandrima, West Bengal State (IN)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/791,086

(22) PCT Filed: Nov. 24, 2005
(86) PCT No.: PCT/IN05/00377

§ 371 Date: May 18, 2007

(30) Foreign Application Priority Data
Nov. 24, 2004 (IN) 1253/CHE/2004

(51) Int. Cl. C12Q 1/68; C07K 16/00

(52) U.S. Cl. 435/6; 530/389.1

(57) ABSTRACT

Disclosed herein are highly specific, polyclonal antibodies of individual Xenopus histones H2A, H2B, H3 and H4 expressed in E. coli and their uses thereof. These four polyclonal antibodies are raised in rabbit and tested against highly purified HeLa core histones. Significantly all of these antibodies are able to identify individual members of the core-histones with high specificity. Thus these antibodies will be very useful tools in studying the in vivo status of the histones during several cellular processes.

This invention relates to novel, individual, polyclonal histone antibodies useful against individual Xenopus histones H2A, H2B, H3 and H4 expressed in E. coli, and application of the said antibodies for diagnostic purposes and as a control in diagnosis.

Antibodies play a pivotal role in the modem medicine. An antibody is a protein synthesized by an animal in response to the presence of a foreign substance called an antigen. Antibodies normally functions to protect the animal from infections. Antibody production in laboratory animals helps obtain high titer, high affinity antisera that can be used in experimentation or diagnostic tests. There are two groups of antibodies; monoclonal antibodies that are the result of a single B-cell—cancer-cell fusion, that has grown up into a clonal population (thus monoclonal) and polyclonal antibodies which are actually a mix of different antibodies (poly (many)—clonal). In the usual case, polyclonals are generated by immunizing an animal, removing the “immune serum” and purifying the immunoglobulin-containing fraction. These antibody mixes recognize several different epitopes since they are generated by a whole pool of B cells, rather than a single one. The difference between the two can be summarized by the fact that monoclonal antibodies are specific to a single target, while polyclonal antibodies have multiple specificities. Polyclonal antibodies are often the preferred choice for detecting denatured proteins or splice variants.

Antibodies have a wide variety of applications such as immunohistochemistry, radioimmunoassay, enzyme-linked immunoabsorbent assay, in therapeutics, as affinity reagents in protein purification, to detect a protein after fractionation by SDS-PAGE, immunoprecipitations. The recent developments in solid phase synthesis of peptides enable the extension of the realm of application of polyclonal antibodies into areas previously thought to be exclusive to monoclonal antibodies. It is now relatively straightforward to produce monospecific polyclonal antibodies via affinity purification using short peptide antigens representing a single epitope. These peptides can also be used to generate very specific immune responses in hosts and provide the basis for many new diagnostic reagents.

The identification and subsequent sequencing of human genes is a major goal of modem scientific research. A large number of valuable human gene products have been synthesized by scientists upon the identification of genes. These include, but are not limited to, human insulin, interferon, Factor VIII, tumor necrosis factor, human growth hormone, tissue plasminogen activator, and numerous other compounds. In addition, knowledge of gene sequences can provide the key insight into the treatment or cure of genetic diseases (such as muscular dystrophy and cystic fibrosis).

Eukaryotic genome is organized in a highly dynamic, complex nucleo-protein structure, chromatin, which consists of DNA-associated with histones and several nonhistone chromatin-associated proteins. A gradual order of complexity leading to the higher ordered folded chromatin fibre begins with the simplest unit of Nucleosome Core Particle (NCP) which has 147 bp of DNA wrapped around a core histone octamer, H3-H4 tetramer and H2A-H2B dimer. At the dyad axis histone HI is positioned and plays a crucial role in higher ordered chromatin fibre formation (1). The structure and function of the chromatin is regulated by post-translational modifications of the histones and nonhistone proteins and ATP-dependent chromatin remodeling machinery. The post-translational modifications (acetylation, phosphorylation, methylation, ubiquitination and sumoylation) of histones and nonhistone chromatin components play a major role in genome function.

Accordingly, it is an object of the present invention to provide novel, individual, polyclonal histone antibodies, which are indeed useful tools for studying in vivo status of histone during several vital cellular processes.

It is a further object of the present invention is to provide diagnostic and therapeutic compositions comprising the said novel antibody along with known additives and adjuvants and also diagnostic tools such as an assay, probe or compositions for studying in vivo status of histone during several vital cellular processes comprising highly specific, polyclonal antibodies raised against individual Xenopus histones H2A, H2B, H3 and H4 expressed in E. coli

The antibodies are raised against individual Xenopus histones H2A, H2B, H3 and H4 expressed in E. coli. These four polyclonal antibodies are raised in rabbit and tested against highly purified HeLa core histones. The purified recombinant protein from E. Coli was injected to the animal as per the known protocols for a definite time interval. Thereafter the antibody titer was checked and the boosted dose of the antigen was given as per the requirement. It should be noted that the Histones are very close in their sequence similarity because of which it is difficult to raise polyclonal antibody specific to each histone. However, the antibody that has been raised and described in the present invention are highly specific and should be very useful for both experimental and diagnostic purposes.

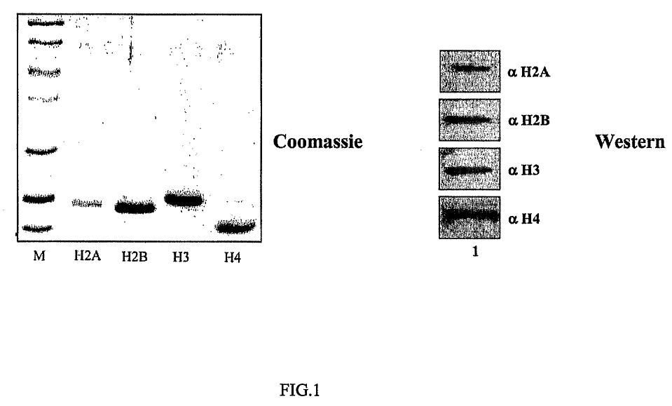

1. Significantly these antibodies are able to identify individual members of the core histones with absolute specificity (FIG. 1). Further from in vivo purified chromatin fractions, these antibodies can efficiently identify all the core histones.

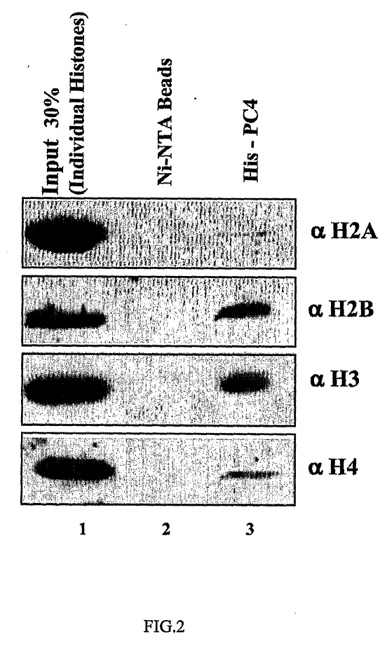

2. The individual histone antibodies are also used to study the histone interaction ability of specific proteins. Recently our lab has shown that human Positive Coactivator PC4 is a chromatin-associated protein showing selective histone interaction ability. PC4-histone interactions are studied in vitro (with purified individual histones or mononucleosomes) and in vivo using these antibodies. These interactions are confirmed by western blotting analysis probing with individual histone antibodies (FIG. 2). Furthermore, the histone interaction ability of PC4 was also verified in vivo using the wholesale extract prepared from He La Cells. PC4 bound histone was pulled down by the antibody against PC4 and the interactive histones were identified by the antibodies of the present invention.

3. In addition immunofluorescence study using anti histone H3 antibodies identifies histone H3 as a nuclear marker.

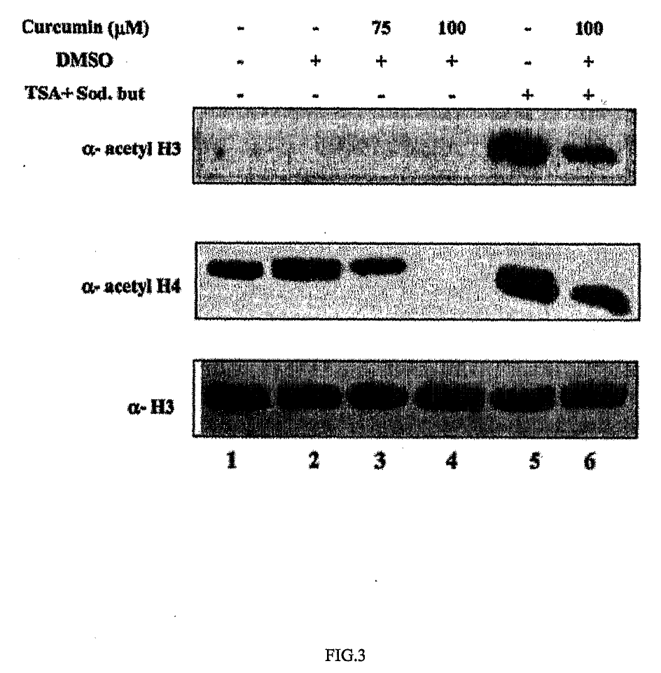

4. Further these antibodies can be also used for in vivo histone modification studies. In experiments for visualizing the different modification status of his tones (like acetylation), these antibodies are used as controls for equal loading of samples. For the functional studies with HAT modulator Curcumin, a difeluromethane, a major curcuminoid in the spice turmeric as a potent p300-specific HAT inhibitor (2), acetylation status of the histones have been probed by anti-Acetylated H3 and H4 antibodies. In these experiments for showing equal loading of the samples anti histone H3 antibodies are used (FIG. 3).

5. These antibodies can also be used in combination with other antibodies for the Chromatin Immunoprecipitation Assays.

This invention will now be described with reference to the accompanying drawings, wherein:

FIG. 1 illustrates the Coomassie and western blotting analysis of individual histones;

FIG. 2 illustrates individual histone interaction with PC4 as probed by anti-histone antibodies; and

FIG. 3 illustrates acetylation status of acid extracted histones probed by anti-acetylated H3 and H4 antibodies with loading of equal amount of histones confirmed by immunodetection of histone H3.

### FIG. LEGENDS

FIG. 1: Coomassie and western blotting analysis of individual histones. For the western blotting analysis α-H2A, α-H2B, α-H3 and α-H4 antibodies have been used.

FIG. 2: The in vitro interactions were assessed by incubating 1 μg of His6-PC4 bound to Ni-NTA beads with individual recombinant core histones, H2A, H2B, H3 and H4, the complexes were pull down and analyzed by western blotting. Lane 1, individual histones; lane 2, the histones incubated with only Ni-NTA agarose; and lane 3, individual histone incubated with Ni-NTA agarose bound to His6-PC4.

FIG. 3: The acid-extracted histones were resolved over 12% SDS-PAGE and were analyzed by western blot using antibodies against acetylated histone H3 and H4. Loading and transfer of equal amounts of protein were confirmed by immunodetection of histone H3. Lane 1, histones extracted from untreated cells, lane 2, DMSO (solvent control) treated cells, lane 3 curcumin (75 μM) treated cells, lane 4, curcumin (100 μM) treated cells, lane 5, trichostatin (2 μM) and sodium butyrate (10 mM) treated cells and lane 6, trichostatin A (2 μM), sodium butyrate (10 mM) and curcumin (100 μM) treated cells are shown.

Therefore the individual histone antibodies raised by the present invention are indeed useful tools for studying in vivo status of histone during several vital cellular processes.

### REFERENCES

1. Wolffe A P, Ihochbin S, Dimitrov S. (1997) What do linker histones do in chromatin, Bioessays, 19, 249-55.

2. Balasubramanyam K, Varier R A, Altaf M, Swaminathan V, Siddappa N B, Ranga U, Kundu T K. (2004) Curcumin, a novel p3001CBP specific inhibitor of acetyltransferase, represses the acetylation of histones/nonhistone proteins and HAT dependent chromatin transcription J. Biol. Chem. 279,51163-71.

1. Highly specific, polyclonal antibodies against individual Xenopus histones H2A, H2B, H3 and H4 expressed in E. coli.

2. The antibodies as claimed in claim 1, which are raised in rabbit and tested against highly purified HeLa core histones.

3. A composition for identifying the individual members of core histones with absolute specificity comprising highly specific, polyclonal antibodies raised against individual Xenopus histones H2A, H2B, H3 and H4 expressed in E. coli.

4. A method for identifying the individual members of core histones with absolute specificity by using a composition containing in vivo purified chromatin fractions of the highly specific, polyclonal antibodies raised against individual Xenopus histones H2A, H2B, H3 and H4 expressed in E. coli.

5. A composition to determine the histone interaction ability of specific proteins comprising highly specific, polyclonal antibodies raised against individual Xenopus histones H2A, H2B, H3 and H4 expressed in E. coli.

6. A diagnostic composition to conduct the in vivo histone modification studies comprising highly specific, polyclonal antibodies raised against individual Xenopus histones H2A, H2B, H3 and H4 expressed in E. coli along with an additive or adjuvant.

7. A diagnostic composition as claimed in claim 7 to conduct studies with HAT modulator Curcumin, a difluromethane, a major curcuminoid in the spice turmeric as a potent p300-specific HAT inhibitor using a composition containing anti-Acetylated H3 and H4 antibodies as probes for histones.

8. A Chromatin Immunoprecipitation Assay comprising highly specific, polyclonal antibodies raised against individual Xenopus histones H2A, H2B, H3 and H4 expressed in E. coli in combination with other antibodies.

9. A diagnostic tool, such as an assay, a probe, a composition for studying in vivo status of histone during several vital cellular processes comprising highly specific, polyclonal antibodies raised against individual Xenopus histones H2A, H2B, H3 and H4 expressed in E. coli
